(19) United States
(12) Patent Application Publication
(10) Pub. No.: US 2023/0270797 A1
(43) Pub. Date: Aug. 31, 2023

(54) NOVEL STRAIN OF THE SPECIES LACTICASEIBADLLUS RHAMNOSUS, COMPOSITIONS THEREOF AND USE THEREOF IN THE TREATMENT OF GENITOURINARY INFECTIONS

(71) Applicant: URIACH ITALY S.R.L., Assago (Mi) (IT)

(72) Inventors: Lorenzo SECONDINI, Assago (Mi) (IT); Cinzia Lucia RANDAZZO, Assago (Mi) (IT); Antonio CIANCI, Assago (Mi) (IT); Stefano CIANCI, Assago (Mi) (IT); Cinzia CAGGIA, Assago (Mi) (IT); Alessandra PINO, Assago (Mi) (IT); Mietta CATERA, Assago (Mi) (IT)

(21) Appl. No.: 18/014,736

(22) PCT Filed: Jul. 9, 2021
(86) PCT No.: PCT/IB2021/056187

§ 371 Date: Jan. 5, 2023

(30) Foreign Application Priority Data
Jul. 9, 2020 (IT) 102020000016666

(51) Int. Cl. A61K 35/744; C12N 1/20; A61P 31/04; A61P 31/10; A61P 15/02

(52) U.S. Cl. CPC A61K 35/744; C12N 1/205; A61P 31/04; A61P 31/10; A61P 15/02; C12R 2001/01

(57) ABSTRACT

The present invention relates to a novel isolated and characterised bacterial strain belonging to the species Lacticas- eibacillus rhamnosus, to compositions thereof and to the use thereof in the treatment of genitourinary infections and/or inflammations

The present invention relates to a novel isolated and characterised bacterial strain belonging to the species Lacticaseibacillus rhamnosus, recently reclassified (Zheng et al., 2020), previously classified as Lactobacillus rhamnosus, to compositions thereof and to the use thereof in the treatment of genitourinary infections and/or inflammations.

The presence of beneficial bacterial strains—both in the intestinal tract and in the genitourinary tract—in appropriate amounts allows to maintain the homeostasis conditions of the microbiota, and protection against opportunistic pathogenic agents. Pathogens responsible for the genitourinary dysbiosis originate in the intestinal tract and, under conditions of impaired defence mechanisms, they are capable of colonising the periurethral region, the bladder and the vagina causing dysbiosis.

Genitourinary dysbiosis are a widespread problem, in particular among the female population.

In most cases they are mixed forms, which originate from a change in the normal vaginal ecosystem and they are characterised by a decrease in lactobacilli and the resulting quantitative growth of opportunistic pathogens such as aerobic bacteria, anaerobic bacteria and yeasts.

The therapy for genitourinary infections in the prior art, in particular for the treatment of vaginal infections such as bacterial vaginitis and vulvovaginal candidiasis, is substantially based on the use of antibiotics or antimycotics. However, antibiotic and antimycotic treatments reveal various disadvantages. In particular, the antibiotic therapy can be associated with side effects of variable nature and severity including: diarrhoea, skin reactions or hypersensitivity to the active ingredient, disorders in the gastroenteric tract. Furthermore, periodic use of antibiotics is associated with the development of resistance and high rate of relapse. Lastly, the antibiotics used commonly are not always effective given that many forms of vaginitis are mixed, caused both by bacteria and by yeasts and—therefore—the antibiotic therapy is non-specific and it often determines further vaginal microbial imbalance.

The use of probiotic bacterial strains for the treatment of genitourinary infections (or genitourinary dysbiosis), in particular the administration of lactobacilli which—as known—play an essential role in the maintenance of the homeostasis conditions of the vaginal microbiota, has been proposed in recent years alternatively to treatments with antibiotics or antimycotics. However, to date, known probiotic bacterial strains are not always effective.

As a matter of fact, it should be observed that the anti-microbial properties of the probiotics are highly variable not only from species to species, but also from strain to strain (strain-specific antimicrobial efficacy).

Pino et. al, (Detection of vaginal lactobacilli as probiotic candidates, Scientific report, March 2019) observed that the balance of vaginal microbiota is dynamic and specific probiotic strains can be used to facilitate the maintenance of the health condition. However, the authors observed that the effect of the probiotics is strain-dependent, and which cannot be transferred from one strain to another, even if belonging to the same species. Similar conclusions are found in US2013/0171253, where there are identified 12 bacterial strains, and their beneficial effects on the vaginal microbiota was evaluated. The strain-dependent effect of probiotics was demonstrated in the study by Mezzasalma et al. (Orally administered multispecies probiotic formulations to prevent uro-genital infections: a randomized placebo-controlled pilot study, general gynecology, 2016).

Furthermore, said probiotic bacterial strains of the prior art recommended for the treatment of genitourinary infections often do not have the characteristics required for the oral administration thereof.

Therefore, there is strongly felt a need for identifying novel treatments for the genitourinary tract infections and/or inflammations alternative to conventional antibiotic or antimycotic treatments, which are highly effective against a broad spectrum of pathogens both of bacterial and fungal nature, well-tolerated, free of significant side effects and effective both through the oral route and through the topical route. Furthermore, there is felt a need for identifying novel treatments capable of reducing or eliminating the problem related to relapse.

Following an intense and extensive research and development activity, with the aim of overcoming the technical problem outlined above, the Applicant isolated (from a healthy female subject), identified and characterised a specific bacterial strain of the genus Lacticaseibacillus belonging to the species Lacticaseibacillus rhamnosus and identified as Lacticaseibacillus rhamnosus TOM 22.8 DMS 33500 (in short, bacterial strain of the invention). Furthermore, the Applicant identified compositions comprising the bacterial strain of the invention (in short, compositions of the invention), advantageously formulated for oral and/or topical administration.

The bacterial strain of the present invention, and as a result the compositions thereof, showed to be capable of simultaneously inhibiting, both in vitro and in vivo, bacteria and yeasts responsible for the main genitourinary infections as well as to have anti-inflammatory and antioxidant activity. Furthermore, said bacterial strain has the technical characteristics to be administered as such or in the form of a composition according to the invention through the oral route, as well as through the topical route.

The bacterial strain of the present invention and the compositions thereof do not have significant side effects and they can be administered to all categories of subjects in need, including pregnant or breastfeeding women, or subjects with other comorbidities.

Lastly, the bacterial strain of the present invention and the compositions thereof are easy to prepare and cost-effective.

These and other objects which will be clear from the detailed description that follows, are attained by the bacterial strain and by the compositions of the present invention due to the technical characteristics claimed in the attached claims.

### FIGURES

FIG. 1: PFGE profile of the genomic DNA of the strain TOM 22.8 (DSM 33500).

FIG. 2: microbial count values expressed as log cfu/ml of vaginal exudate at the time of recruitment (TO), after 10 days (T1) and after one month from the oral and topical treatment with the strain TOM22.8.

### DETAILED DESCRIPTION OF THE INVENTION

Forming an object of the present invention is a bacterial strain of the genus Lacticaseibacillus belonging to the species Lacticaseibacillus rhamnosus identified as Lacticaseibacillus rhamnosus TOM 22.8 and deposited at Deutsche Sammlung von Mikroorganismen and Zellkulturen GmbH (DSMZ) under deposit number DSM 33500 by URIACH ITALY Srl on 17 Apr. 2020 (in short, bacterial strain of the invention or Lacticaseibacillus rhamnosus TOM 22.8 DSM 33500).

The bacterial strain Lacticaseibacillus rhamnosus TOM 22.8 DSM 33500 has functional and safety technical characteristics as reported in the experimental part. In particular, the bacterial strain Lacticaseibacillus rhamnosus TOM 22.8 DSM 33500 has shown an antagonistic activity against a broad spectrum of bacteria and yeasts, for example against Gardnerella vaginalis, Escherichia coli, Staphylococcus aureus, Candida albicans, Candida krusei, Candida glabrata, and Candida tropicalis; anti-inflammatory and antioxidant activity; ability to adhere to the intestinal mucosa; ability to produce exopolysaccharides (EPS); good freeze-drying ability and stability; optimal characteristics that allow the administration thereof through the oral route (gastroenteric).

Furthermore, the bacterial strain Lacticaseibacillus rhamnosus TOM 22.8 DSM 33500 is capable of restoring the microbiota balance of the genitourinary tract by increasing the number of lactobacilli and thus exerting an inhibitory action against opportunistic pathogens. As a matter of fact, there is generally observed the proliferation of opportunistic pathogen microorganisms in the microbiota of the genitourinary tract as a result of a decrease in lactobacilli (dysbiosis).

Forming an object of the present invention is a mixture comprising or, alternatively, consisting of a bacterial strain of the genus Lacticaseibacillus belonging to the species Lacticaseibacillus rhamnosus identified as Lacticaseibacillus rhamnosus TOM 22.8 and deposited at Deutsche Sammlung von Mikroorganismen and Zellkulturen GmbH (DSMZ) under deposit number DSM 33500 by URIACH ITALY Srl on 17 Apr. 2020 (in short, bacterial strain of the invention or Lacticaseibacillus rhamnosus TOM 22.8 DSM 33500).

Forming an object of the present invention is a composition (in short, composition of the invention) comprising or, alternatively, consisting of said bacterial strain Lacticaseibacillus rhamnosus TOM 22.8 DSM 33500 and at least one acceptable pharmaceutical or food grade additive and/or excipient.

Said bacterial strain was deposited according to the provisions pursuant to the Budapest Treaty; the depositing party of said bacterial strain described and claimed in the present patent application and the proprietor thereof express the consent thereof to make the strain available over the entire duration of the patent.

The bacterial strain Lacticaseibacillus rhamnosus TOM 22.8 DSM 33500, as such or comprised in the composition of the invention, according to any one of the described embodiments, may be a viable bacterial strain (or probiotic strain) or a derivative of said strain, wherein said derivative is capable of exerting a similar beneficial activity on the health of the host of the corresponding viable bacterial strain. Preferably, the strain Lacticaseibacillus rhamnosus TOM 22.8 DSM 33500, as such or comprised in the composition of the invention, according to any one of the described embodiments, is a viable bacterial strain (or probiotic strain).

“Probiotics” (e.g. bacterial strains) are live and viable microorganisms which, when administered in adequate amount, confer benefits to the health of the host (FAO/WHO definition, 2002); the term “probiotic” refers to microorganisms present in or added to food or administered in the form of capsules, tablets or etc.

In the context of the present invention, the expression “derivative” of the bacterial strain (or “derivative” of the strain) is used to indicate the tyndallized or sonicated bacterial strain, the lysates of bacterial strains, the extracts or the parietal fraction of the bacterial strain (referred to as paraprobiotics), the metabolites or metabolic bioproducts or exopolysaccharides (EPS) generated by the bacterial strain (referred to as postbiotics) and/or any other product derived from bacterial strain known to the person skilled in the art.

Said derivatives are obtained according to methods known to the person skilled in the art. Preferably the expression “derivative” of the bacterial strain is used to indicate the tyndallized strain Lacticaseibacillus rhamnosus TOM 22.8 DSM 33500.

In the context of the present invention, the acceptable pharmaceutical or food grade additives and/or excipients comprise all the ancillary substances known to the person skilled in the art for the preparation of compositions in solid, semi-solid or liquid form, such as for example carriers, diluents, solvents solubilisers, acidifiers, thickeners, sweeteners, flavour-enhancement agents, colorants, sweeteners, lubricants, surfactants, preservatives, stabilisers, pH stabilising buffers and mixtures thereof.

The composition of the invention comprising the strain Lacticaseibacillus rhamnosus TOM 22.8 DSM 33500, according to any one of the described embodiments, may further comprise at least one prebiotic, preferably selected from: a non-starch polysaccharide or beta-glucan, a fructan, a fructooligosaccharide (FOS), galactooligosaccharide (GOS), xylooligosaccharide (XOS), an inulin, a pyrodextrin, soybean oligosaccharides; more preferably selected from: inulin, fructooligosaccharide (FOS), galactooligosaccharide (GOS), xylooligosaccharide (XOS), guar gum, and mixtures thereof.

The composition of the invention, comprising the strain Lacticaseibacillus rhamnosus TOM 22.8 DSM 33500 and, optionally, a prebiotic (e.g. inulin), may further comprise at least one or more further probiotic bacterial strains or derivatives thereof, preferably one or more further strains belonging to the family Lactobacillaceae, more preferably one or more further strains belonging to the family Lacticaseibacillus rhamnosus.

The composition of the invention, comprising the strain Lactobacillus rhamnosus TOM 22.8 DSM 33500 and, optionally, a prebiotic (e.g. inulin) and/or at least one further probiotic bacterial strain or a derivative thereof, may further comprise at least one further active component, preferably adjuvant for the treatment of a dysbiosis/genitourinary infection and/or inflammation, selected from: a plant product (botanicals), or the extract or derivative thereof, such as for example cranberry (Vaccinum Macrocarpon), grapefruit seeds, hibiscus, sage, thyme, ginger, turmeric, bearberry (Arctostaphylos uva-ursi); an essential oil, such as for example tea tree oil; a vitamin of group B, C, D, A or E; an antioxidant substance, for example glutathione, a polyphenol (e.g. resveratrol), coenzyme Q10, astaxanthin or lipoic acid; a mineral, for example zinc, magnesium or manganese-copper; D-mannose, lactoferrin, L-methionine, hyaluronic acid, glucosamine, chondroitin, collagen, a substance with oestrogenic activity, and mixtures thereof.

The composition of the invention, comprising the strain Lacticaseibacillus rhamnosus TOM 22.8 DSM 33500 and, optionally, a prebiotic and/or a further probiotic and/or a further active component, according to any one of the described embodiments, may further comprise said at least one acceptable pharmaceutical or food grade additive and/or excipient, that is a substance devoid of therapeutic activity suitable for pharmaceutical or food use. In the context of the present invention the additives and/or excipients acceptable for pharmaceutical or food use comprise all ancillary substances known to the person skilled in the art for the preparation of compositions in solid, semi-solid or liquid form, such as for example diluents, solvents (including water, glycerine, ethyl alcohol), solubilisers, acidifiers, thickeners, sweeteners, flavour-enhancement agents, dyes, lubricants, surfactants, preservatives, stabilisers, pH stabilising buffers and mixtures thereof.

Said compositions of the invention, according to any one of the described embodiments, may be a pharmaceutical composition (or Live Biotherapeutic Products), a medical device composition, a dietary supplement, a food (or novel food or food for special medical purposes), a composition for a dietary supplement or food, or, alternatively, a composition for cosmetic use.

The compositions of the present invention, according to any one of the described embodiments, can be formulated for oral use, for topical use, such as topical vaginal or genital use, for rectal use, for nasal inhalation (e.g. spray or drops), for oral inhalation (e.g. spray, dry inhalation powder). Preferably, the compositions of the present invention are for oral or topical vaginal/genital use.

In the context of the present invention, the expression for oral use is used to indicate both oral (or gastroenteric) administration and sublingual (or buccal) administration.

The composition of the present invention may be formulated for oral use in solid form, for example, selected from: tablets, chewable tablets, mouth-dissolving tablets, capsules, granules, flakes, powder, soluble powder, mouth-dissolving powder; or, alternatively, in liquid form, for example, selected from:

solutions, suspensions, emulsions, liquid which can be dispensed in the form of spray, syrups; or, alternatively, in semi-liquid form, for example, selected from: soft-gel, gel; preferably the composition of the invention is for oral use in solid form.

Alternatively, the composition of the present invention may be formulated for topical use, preferably topical vaginal or genital use, or for rectal use in solid form selected from: ovules, pessaries, capsules, suppositories; or, alternatively, in liquid form selected from: solutions, suspensions or emulsions for vaginal washes (douches); or, alternatively, in semi-liquid form selected from: gel, foam or cream.

Forming an object of the present invention is the strain Lacticaseibacillus rhamnosus TOM 22.8 DSM 33500 and the compositions thereof, according to any one of the described embodiments, for use as medicament.

Forming an object of the present invention is Lacticaseibacillus rhamnosus TOM 22.8 DSM 33500 and the compositions thereof, according to any one of the described embodiments, for use in a method for preventive and/or curative treatment of dysbiosis, genitourinary tract infections and/or inflammations (or genital and/or urinary tract), and of associated disorders or symptoms in a subject in need (i.e. a mammal), preferably a male or female human subject, more preferably a female human subject; wherein said treatment method comprises the administration of a therapeutically effective amount of said bacterial strain of the present invention or of a composition thereof to the subject.

Forming an object of the present invention is a mixture comprising or, alternatively, consisting of a bacterial strain of the genus Lacticaseibacillus belonging to the species Lacticaseibacillus rhamnosus identified as Lacticaseibacillus rhamnosus TOM 22.8 and deposited at Deutsche Sammlung von Mikroorganismen und Zellkulturen GmbH (DSMZ) under deposit number DSM 33500 by URIACH ITALY Srl on 17 Apr. 2020 (in short, bacterial strain of the invention or Lacticaseibacillus rhamnosus TOM 22.8 DSM 33500).

Said mixture may further comprise pharmaceutical or food grade technological additives. An example of said technological additives may for example be cutting additives, usually used in this technical field, and known to the person skilled in the art.

The mixture as intended in this context may solely consist of the active ingredient, bacterial strain of the genus Lacticaseibacillus belonging to the species Lacticaseibacillus rhamnosus identified as Lacticaseibacillus rhamnosus TOM 22.8 and deposited at Deutsche Sammlung von Mikroorganismen und Zellkulturen GmbH (DSMZ) under deposit number DSM 33500 by URIACH ITALY Srl on 17 Apr. 2020 (in short, bacterial strain of the invention or Lacticaseibacillus rhamnosus TOM 22.8 DSM 33500). Alternatively, said mixture may comprise said strain and other bacterial strains, and/or pharmaceutical or food grade technological additives.

The mixture as described herein may be advantageously used in the treatment of dysbiosis, genitourinary tract dysbiosis/infections and/or inflammations, preferably of the genital tract, and the associated disorders or symptoms are selected from: bacterial and/or fungal vaginitis, vaginal (or vulvovaginal) candidiasis , bacterial vaginitis, atrophic vaginitis, male genital bacterial and/or fungal infections, balanitis, posthitis and balanoposthitis, pain and/or itching in the genital area, dysuria, strangury, malodorous vaginal discharge, vaginal dryness, and dyspareunia; wherein said infections and/or inflammations may be acute or chronic.

According to a first aspect of the present invention, said dysbiosis/genitourinary tract infections and/or inflammations, preferably of the genital tract, and the associated disorders or symptoms are selected from: bacterial and/or fungal vaginitis, vaginal (or vulvovaginal) candidiasis , bacterial vaginitis, atrophic vaginitis, male genital bacterial and/or fungal infections, balanitis, posthitis and balanoposthitis, pain and/or itching in the genital area, dysuria, strangury, malodorous vaginal discharge, vaginal dryness, and dyspareunia; wherein said infections or inflammations may be acute or chronic.

Advantageously, for the treatment of atrophic vaginitis, the strain Lacticaseibacillus rhamnosus TOM 22.8 DSM 33500, used as such or in the form of a composition as described in the present invention, may be combined with a substance with oestrogenic activity, for example a phytoestrogen selected from: an isoflavone (e.g. genistein, daidzein, biocaine A, glycitein), a coumestan (e.g. coumestrol and methoxycoumestrol), a lignan (e.g. enterodiol, enterolactone), a derivative from a plant (e.g. soy, red clover, legumes), and mixtures thereof.

According to a further aspect of the present invention, said genitourinary tract infections and/or inflammations, preferably of the urinary tract, and associated disorders or symptoms are selected from: bacterial and/or fungal urinary tract infections (UTI) or of the lower urinary tract, cystitis, urethritis, prostatitis, lower urinary tract infections in subjects with bladder catheters, pain and/or itching in the genital area, dysuria and painful urination; wherein said infections and/or inflammations may be acute or chronic.

Advantageously, for the treatment of cystitis, the strain Lacticaseibacillus rhamnosus TOM 22.8 DSM 33500, used as such or in the form of a composition as described in the present invention, may be combined with a substance, or extracts and/or derivatives thereof, selected from: cowberry, cranberry (Vaccinum Macrocarpon), grapefruit seeds, bearberry (Arctostaphylos uva-ursi), manganese-copper, essential oil tea tree oil, or mixtures thereof.

The bacterial strain of the present invention and the compositions thereof, according to any one of the described embodiments, may be for use in the treatment of the aforementioned dysbiosis, genitourinary tract infections and/or inflammations both as a single therapy and as adjuvant therapy of further pharmacological therapies.

For example, in the treatment of dysbiosis, genitourinary or urinary tract infections and/or inflammations, the bacterial strain of the invention and the compositions thereof may be a support to an antibiotic treatment.

The bacterial strain of the present invention and the mixtures thereof, and compositions, according to any one of the described embodiments, may be for therapeutic, preventive and/or curative use, for the decrease or suppression of the onset of relapses of genitourinary tract infections and/or inflammations in a male or female subject in need, preferably in a female subject (e.g. vaginal infections).

Preferably, the strain Lacticaseibacillus rhamnosus TOM 22.8 DSM 33500 and/or the mixtures or compositions thereof, according to any one of the described embodiments, are for use in a method for preventive and/or curative treatment of dysbiosis, vaginal or vulvovaginal tract infections and/or inflammations of bacterial and/or fungal origin, and associated disorders or symptoms, preferably bacterial and/or fungal vaginitis, vaginal (or vulvovaginal) candidiasis and bacterial vaginosis; wherein said composition is formulated for oral or topical vaginal use.

Advantageously, the strain Lacticaseibacillus rhamnosus TOM 22.8 DSM 33500 and the compositions of the present invention, according to any one of the described embodiments, are for use in a method for preventive and/or curative treatment of dysbiosis, genitourinary tract infections and/or inflammations, preferably of the vagina, caused by aerobic and/or anaerobic bacteria and/or yeasts commonly found in lower genitourinary tract infections, such as for example Gardnerella vaginalis, Escherichia coli, Staphylococcus aureus, Candida albicans, Candida krusei, Candida glabrata, Candida tropicalis.

As a matter of fact, the strain Lacticaseibacillus rhamnosus TOM 22.8 DSM 33500 showed antimicrobial activity against: Gardnerella vaginalis, Escherichia coli, Staphylococcus aureus, Candida albicans, Candida krusei, Candida glabrata, and Candida tropicalis; in particular, against Escherichia coli ATCC 700414, Escherichia coli ATCC 25922, Staphylococcus aureus ATCC 6538, Gardnerella vaginalis ATCC 14018, Gardnerella vaginalis ATCC 14019, Candida albicans ATCC 10231, Candida krusei ATCC 14243, Candida glabrata ATCC 90030, and Candida tropicalis ATCC 13803.

Vaginitis is an acute or chronic inflammation of the vagina caused by an infection often linked to a change in the normal balance of the vaginal microbiota (vaginal dysbiosis). The main causes of vaginitis are bacterial infections (bacterial vaginosis) and yeast infections (fungal vaginitis or vaginal candidiasis). Vaginitis are often caused by a mixed population of aerobic and anaerobic bacteria (e.g. Gardnerella vaginalis) and yeasts (e.g. Candida albicans and/or Candida spp.) that coexist and contribute to the development of the infection. Furthermore, low oestrogen levels after menopause may cause a vaginitis referred to as atrophic vaginitis. Besides local disorders, untreated vaginal infections may lead to further complications. For example, bacterial vaginosis may cause premature delivery or postpartum infections for pregnant women, pelvic inflammatory disease, post-surgical complications (after abortion, hysterectomy or caesarean section), increased vulnerability to HIV infection or sexually transmitted diseases (chlamydia, mycoplasma, human papilloma virus, herpes virus) which may cause infertility or cervical cancers due to some oncogenic strains of human papilloma virus.

In the context of the present invention, the expression dysbiosis is used to indicate a microbial imbalance (e.g. of bacteria and yeasts) on a surface or mucosa of the body.

Bacterial vaginosis (BV) is a vaginal disease caused by an excessive growth of opportunistic pathogenic bacteria derived from the vaginal ecosystem. Bacterial vaginosis infection in a subject increases the risk of contracting further sexually transmitted infections, including HIV, and preterm delivery.

Vaginal (or vulvovaginal) candidiasis is the fungal infection resulting from the uncontrolled proliferation, at the level of the vagina, of a yeast of the spades Candida, in particular of Candida albicans.

Balanitis is an inflammation of the glans, the posthitis of the foreskin and the balanoposthitis of both.

According to an aspect of the invention, the bacterial strain Lacticasibacillus rhamnosus TOM 22.8 DSM 33500 and the compositions thereof, according to any one of the described embodiments, are for use in a treatment method for colonisation with Lactobacilli from the mucosa of the genitourinary tract (e.g. vagina) and/or to prevent or inhibit the colonisation and/or the growth of pathogens (e.g. opportunistic pathogens) in said genitourinary tract, and/or to restore the microbiota of said genitourinary tract in a male or female subject in need, preferably in a female subject, for example, in cases of dysbiosis and/or after severe medical treatments such as those with antibiotics or radiations.

Advantageously, in order to be effective in the treatment methods described in the present invention, said bacterial strain Lacticaseibacillus rhamnosus TOM 22.8 DSM 33500 is administered as such or as a composition of the invention to a subject in need in an oral and/or topical daily dose comprising said bacterial strain in an amount comprised in the range from 10×10⁶ cfu to 10×10¹¹ cfu, preferably from 10×10⁸ cfu to 10×10¹⁰ cfu, more preferably in a concentration of about 10×10⁸ cfu or 10×10⁹ cfu (cfu: Colony Forming Unit).

The aforementioned daily doses may be administered to the subject in need in a single dose (one dose) or in repeated doses, for example two, three or four daily doses.

Unless specified otherwise, the expression composition or other comprising a component at an amount “comprised in a range from x to y” is used to indicate that said component may be present in the composition or other at all the amounts present in said range, even though not specified, extremes of the range comprised.

Unless specified otherwise, the indication that a composition “comprises” one or more components or substances means that other components or substances can be present besides the one, or the ones, indicated specifically.

In the context of the present invention, the expression “treatment method” is used to indicate an intervention on a subject in need, comprising the administration of the bacterial strain or of a composition of the invention with the aim of eliminating, reducing/decreasing or preventing a disease or ailment and the symptoms or disorders thereof.

The expression “subject/s” in the context of the present invention is used to indicate mammals, preferably female or male human subjects, more preferably female human subjects.

The expression “therapeutically effective amount” refers to the amount of active compound or bacterial strain that elicits the biological or medicinal response in a tissue, system, mammal, or human being that is sought and defined by an individual, researcher, veterinarian, physician, or other clinician or health worker. In the context of the present invention, the expression “medical device” is used in the meaning according to the Legislative Decree n° 46 dated 24 Feb. 1997, or in accordance with the new Medical device regulation (EU) 2017/745 (MDR).

In the context of the present invention, the term “novel food” is used in the meaning according to Regulation EC 258 dated 1997.

An example of a composition according to the invention is reported in Table 1.

TABLE 1
10B: 10 billion.

| Component | mg/capsule |
|---|---|
| Probiotic strain TOM 22.8 | 10B end of shelf life |
| Dried starch | q.s. |
| Stearate magnesium | q.s. |
| Magnesium oxide | q.s. |
| Capsule V PLUS size 0 | 95 |
| TOTAL | 500 |

### EMBODIMENTS

FR1. A bacterial strain belonging to the species Lacticaseibacillus rhamnosus identified as Lacticaseibacillus rhamnosus TOM 22.8 and deposited at Deutsche Sammlung von Mikroorganismen and Zellkulturen GmbH (DSMZ) under deposit number DSM 33500.

l FR 2. A composition comprising or, alternatively, consisting of said bacterial strain according to FR 1 and at least one pharmaceutical or food grade additive and/or excipient.

FR 3. The composition according to FR 2, wherein said composition further comprises at least one prebiotic; preferably said at least one prebiotic is selected from: inulin, fructooligosaccharide (FOS), galactooligosaccharide (GOS), xylooligosaccharide (XOS), guar gum and mixtures thereof; more preferably inulin.

FR 4. The composition according to FR 2 or 3, wherein said composition is formulated for oral use; preferably in solid form.

FR 5. The composition according to any one of FRs 2-4, wherein said composition is formulated for topical use; preferably for topical vaginal or genital use.

FR 6. The composition according to any one of FRs 2-5 or, alternatively, the bacterial strain according to claim 1 for use as medicament.

FR 7. The composition according to any one of FRs 2-5 or, alternatively, the bacterial strain according to claim 1 for use in a method for the preventive or curative treatment of a genitourinary infection and/or inflammation, and of associated disorders or symptoms, in a male or female subject in need; preferably in a female subject.

FR 8. The composition for use according to FR 7 or, alternatively, the bacterial strain for use according to

FR 7 wherein said genitourinary infection and/or inflammation, preferably of the genital tract, and the associated disorders or symptoms are selected from: bacterial and/or fungal vaginitis, vaginal (or vulvovaginal) candidiasis , bacterial vaginitis, atrophic vaginitis, male genital bacterial and/or fungal infections, balanitis, posthitis and balanoposthitis, pain and/or itching in the genital area, dysuria, strangury, malodorous vaginal discharge, vaginal dryness, and dyspareunia; wherein said infection or inflammation is acute or chronic.

FR 9. The composition for use according to FR 7 or 8 or, alternatively, the bacterial strain for use according to FR 7 or 8, wherein said genitourinary infection and/or inflammation is a vaginal infection and/or inflammation of bacterial and/or fungal origin; preferably selected from bacterial and/or fungal vaginitis, vaginal candidiasis and bacterial vaginosis.

FR 10. The composition for use according to FR 7 or, alternatively, the bacterial strain for use according to FR 7, wherein said genitourinary infection and/or inflammation, preferably of the urinary tract, and the associated disorders or symptoms are selected from: bacterial and/or fungal urinary tract infections (UTI) or of the lower urinary tract, cystitis, urethritis, prostatitis, lower urinary tract infections in subjects with bladder catheters, dysuria and painful urination; wherein said infection or inflammation is acute or chronic.

### EXPERIMENTAL PART

### I. Isolation of the Strain Lacticaseibacillus rhamnosus TOM 22.8 DSM 33500, Phenotypic and Genotypic Characterisation

The strain Lacticaseibacillus rhamnosus TOM 22.8 DSM 33500 was isolated from the vaginal ecosystem of a healthy patient, recruited at the Obstetric and Gynecologic Clinic “Policlinico-Vittorio Emanuele”, University of Catania, using the growth substrate De Man, Rogosa and Sharpe (MRS).

The strain was subjected to phenotypic characterisation: morphological observation under the microscope, Gram staining, catalase test, spore-production capacity and motility evaluation (Table 2).

TABLE 2
Phenotypic characterisation

| | Morphological | | | Spore- | |
|---|---|---|---|---|---|
| | under | Gram | | production | |
| | microscope | staining | Catalase | capacity | Motility |
| TOM 22.8 | Bacillary | Gram | Negative | Absent | Absent |
| | | positive | | | |

The strain was subjected to genotypic characterisation: Tuf-gene species-specific PCR, and strain-level typing through Pulsed Field Gel Electrophoresis (PFGE) analysis, in compliance with the requirements laid down by the FAO/WHO 2002 guidelines (see annex).

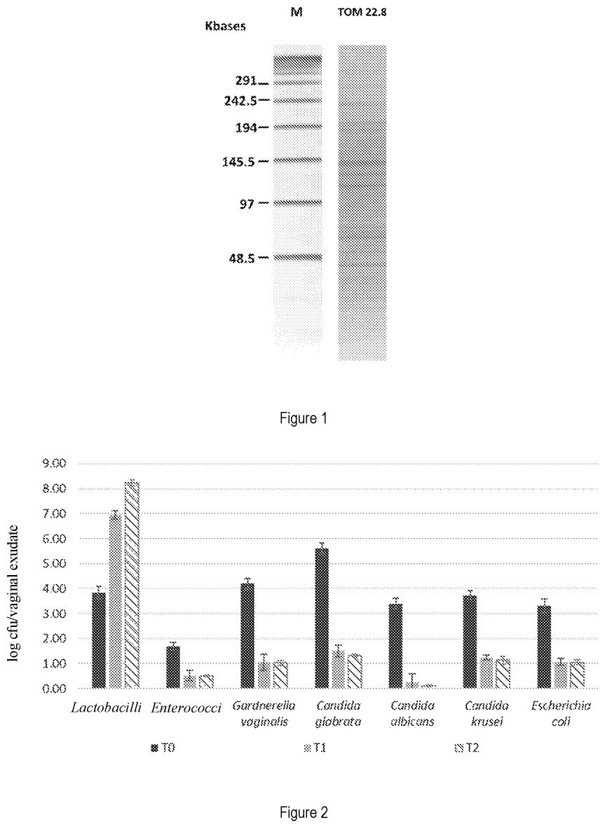

In particular, the strain TOM 22.8 (DSM 33500) was genetically identified through complete genome sequencing, by means of illumine MiSeq analysis. Analysis showed that the strain TOM 22.8 (DSM 33500) belongs to the species Lacticaseibacillus rhamnosus. Furthermore, molecular typing (genetic fingerprinting) of the strain TOM 22.8 (DSM 33500) was carried out by means of pulsed field gel electrophoresis (PFGE). The results of the genomic profile of the strain TOM 22.8 are reported in FIG. 1.

### II. Safety Performance

In accordance with the guidelines for probiotics and prebiotics, drawn up by the Ministry of Health, the strain Lacticaseibacillus rhamnosus TOM 22.8 was evaluated for safety requirements. In detail, the following were studied: haemolytic activity, ability to produce DNAse and gelatinase, bile salt hydrolase (BSH) activity, ability to degrade mucin as well as antibiotic resistance against various antibiotics, regarding which EFSA established break-point values.

S. pyogenes ATCC 19615 and S. pneumoniae ATCC 6303 were respectively used as positive controls for β-haemolysis and α-haemolysis. The strains Streptococcus pyogenes ATCC 19615 and Streptococcus pneumoniae ATCC 6303 were cultured on Brain-Heart Infusion (BHI, Becton Dickinson GmbH, Germany) at 37 ° C. below 5% CO2 and respectively used as positive controls for β-haemolysis and α-haemolysis). The strain L. acidophilus DRU was used as a positive control for the bile salt hydrolase.

In detail below are the characterisations carried out on the strain under study.

### II.1. Haemolytic Activity

The strain Lacticaseibacillus rhamnosus TOM 22.8 DSM 33500 was revitalised in MRS broth medium and incubated at 37° C. for 18-24 hours. The cell culture in exponential growth phase was transferred, by means of propagation smear, into Blood Agar plates containing 5% of defibrinated ram blood (Biolife, Milan, Italy) and incubated at 37° C. for 24-48 hours. Haemolytic activity was visually determined and distinguished as β-haemolysis, α-haemolysis or γ-haemolysis based on the presence of clear zones, green halos or no zone around the colonies, respectively. The β-haemolytic strain Streptococcus pyogenes ATCC 19615 and the α-haemolytic strain Streptococcus pneumoniae ATCC 6303 were used as positive controls. The results, reported in Table 3, were expressed as +(presence of activity) and −(absence of activity). The strain tested showed no haemolytic activity.

TABLE 3

| Activity tested | L. rhamnosus TOM 22.8 |
|---|---|
| Haemolytic activity | − |

### II.2. Ability to Produce DNAse and Gelatinase

DNAse production was tested by transferring 5 pl of a cell culture in exponential growth phase into DNAse agar plates (Oxoid). After incubation at 37° C. for 48 hours, the plates were treated with 1N HCl for 5 minutes. The presence of clear zones around the colonies is considered as an indicator of positive DNAse production.

Gelatinase production was evaluated using gelatine agar plates (30 g/L gelatine, 5 g/L peptone, 3 g/L yeast extract and 17 g/L agar). After incubation at 37° C. for 48 h, the surface of the plates was treated with saturated ammonium sulphate (heck). The presence of clear zones around the colonies is considered as an indicator of positivity of gelatinase activity.

For both tests, the results, reported in Table 4, were expressed as: −(absence of activity), +(presence of activity). The strain tested showed no ability to produce DNAse and gelatinase.

TABLE 4

| Activity tested | L. rhamnosus TOM 22.8 |
|---|---|
| DNAse | − |
| Gelatinase | − |

### II.3. Bile Salt Hydrolase (BSH) Activity

BSH activity was assayed using MRS agar plates added with 0.5% (w/v) taurodeoxycholic acid (TDCA, Sigma). Cell cultures in exponential growth phase were transferred to the surface of the aforementioned growth medium and incubated anaerobically for 48 hours at 37° C. The precipitation of taurodeoxycholic acid around the colonies is considered to be an indicator of the positivity of gelatinase activity. The strain Lactobacillus acidophilus DRU was used as a positive control.

The results, reported in Table 5, were expressed as: −(absence of activity), +(presence of activity). The strain tested showed no bile salt hydrolase ability.

TABLE 5

| Activity tested | L. rhamnosus TOM 22.8 |
|---|---|
| BSH | − |

### II.4. Ability to Degrade Mucin

The ability of the strain Lacticaseibacillus rhamnosus TOM 22.8 DSM 33500 to degrade mucin was evaluated using a suitable modified growth medium by adding 0.5% (w/v) of porcine stomach type III (Sigma-Aldrich Corp.). The cell culture in exponential growth phase was transferred to the aforementioned growth medium and the plates were incubated anaerobically at 37° C. for 72 hours. Subsequently, the plates were stained with 0.1% (w/v) black starch (Merck KGaA) in 3.5 M acetic acid for 30 minutes, then washed with 1.2 M acetic acid (Merck KGaA). The presence of halos around the colonies was considered a positive result.

The results, reported in Table 6, were expressed as: −(absence of activity), +(presence of activity). The strain tested showed no ability to degrade mucin.

TABLE 6

| Activity tested | L. rhamnosus TOM 22.8 |
|---|---|
| Mucin degradation | − |

### II.5. Antibiotic Susceptibility and Determination of MIC

The strain Lacticaseibacillus rhamnosus TOM 22.8 DSM 33500 is considered resistant or sensitive to the antibiotics tested, as reported by the European Food Safety Authority (EFSA), by evaluating the minimum inhibitory concentration (MIC) using the cut-offs recommended by EFSA (2012) as reference. For each antibiotic tested, the strain was classified as resistant (R) or sensitive (S). As observable from the results, reported in Table 7, the strain Lacticaseibacillus rhamnosus TOM 22.8 is sensitive to all the antibiotics tested. Furthermore, the strain did not show antibiotic resistance genes at the genome level. In Table 7, the break-point values for each antibiotic tested are reported in brackets and they are expressed in μg/ml.

TABLE 7

| Activity tested | | TOM 22.8 |
|---|---|---|
| Antibiotic resistance | Ampicillin (4) | 2^{S} |
| | Gentamicin (16) | <4^{S} |
| | Kanamycin (64) | 16^{S} |
| | Streptomycin (32) | 8^{S} |
| | Erythromycin (1) | 1^{S} |
| | Clindamycin (1) | 0.25^{S} |
| | Tetracycline (8) | 4^{S} |
| | Chloramphenicol (4) | 1^{S} |

### III. Functional Performance

### III.1. Resistance to Lysozyme

The ability of the strain Lacticaseibacillus rhamnosus TOM 22.8 DSM 33500 to survive in the presence of lysozyme was evaluated in MRS broth after 0, 30, and 120 minutes incubation at 37° C. Tolerance to lysozyme was determined by plate counting viable cells. The analysis was carried out in triplicate and the results, reported in Table 8 as mean and standard deviation, are expressed as log cfu/ml. Furthermore, the % survival was evaluated, calculated taking into account the final (cfuF) and initial (cful) population density (cful) (cfuF/cful *100) and the value 80% was considered as the minimum survival limit. Cell cultures, of the strain Lacticaseibacillus rhamnosus TOM 22.8 DSM 33500, were used as control in the absence of lysozyme. The strain tested showed ability to survive in the presence of lysozyme, having survival % equal to 94% and 91% after incubation at 37° C. for 30 and 120 minutes respectively.

TABLE 8

| Activity tested | | TOM 22.8 |
|---|---|---|
| Resistance to lysozyme | 0 min | 9.11 ± 0.11 |
| (log cfu/ml) | 30 min | 8.53 ± 0.09 |
| | 120 min | 8.31 ± 0.11 |

### III.2. Tolerance at Low pH Values

The ability of the strain Lacticaseibacillus rhamnosus TOM 22.8 DSM 33500 to tolerate low pH values was tested in MRS broth acidified to pH 2.0 and pH 3.0 by adding 1M HCI MRS at pH 6.2 was used as control. The strain under study was revitalised in MRS broth and the cell suspension in exponential growth phase (9 log cfu/mL) was inoculated into the acidified growth medium. Aliquots were taken immediately after inoculation (0 hours) and after 2 and 4 hours of incubation at 37° C.

Tolerance to low pH values was determined by plate counting viable cells. The analysis was carried out in triplicate and the results, expressed as log cfu/ml, are reported as mean and standard deviation. Furthermore, the % survival was evaluated, calculated taking into account the final (cfuF) and initial (cful) population density (cful) (cfuF/cful *100) and the value 80% was considered as the minimum survival limit. The strain tested showed ability hours to survive at pH 3.0 and 2.0, having a survival % greater than 80% after incubation at 37° C. for 2 hours and 4 hours (Table 9). The survival percentage of at pH 3.0 after 4 hours is equal to 91%. The survival percentage of at pH 2.0 after 4 hours is equal to 92%.

TABLE 9

| Activity tested | | TOM 22.8 |
|---|---|---|
| Resistance at pH 3.0 (log cfu/ml) | 0 h | 9.24 ± 0.13 |
| | 2 h | 8.86 ± 0.09 |
| | 4 h | 8.44 ± 0.06 |
| Resistance at pH 2.0 (log cfu/ml) | 0 h | 9.11 ± 0.05 |
| | 2 h | 8.78 ± 0.10 |
| | 4 h | 8.41 ± 0.07 |

### III.3. Tolerance to Bile Salts

The ability of the strain Lacticaseibacillus rhamnosus TOM 22.8 DSM 33500 to survive in the presence of bile salts (bovine bile salts, Oxgall; Sigma-Aldrich), at final concentrations of 0.5% and 1.0% weight/volume was evaluated as follows. The strain under study was revitalised in MRS broth and the cell suspension in exponential growth phase (9 log cfu/mL) was inoculated in MRS broth containing the percentages of bile salts reported above. MRS without bile salts was used as control. Aliquots were taken immediately after inoculation (0 hours) and after 2 and 4 hours of incubation at 37° C. and the plate count of viable cells was carried out. The analysis was carried out in triplicate and the results, reported in Table 10 and expressed as log cfu/ml, are reported as mean and standard deviation. Furthermore, the % survival was evaluated, calculated taking into account the final (cfuF) and initial (cful) population density (cful) (cfuF/cful *100) and the value 80% was considered as the minimum survival limit.

The strain tested showed ability to survive in the presence of bile salts at concentrations of 0.5% and 1%. The percentage survival of the strain at the various concentrations of bile salts tested was greater than 80%.

TABLE 10

| Activity tested | | TOM 22.8 |
|---|---|---|
| Resistance to bile 0.5% | 0 h | 9.12 ± 0.14 |
| (log cfu/ml) | 2 h | 8.06 ± 0.06 |
| | 4 h | 7.50 ± 0.05 |
| Resistance to bile 1% | 0 h | 9.12 ± 0.04 |
| (log cfu/ml) | 2 h | 8.16 ± 0.05 |
| | 4 h | 8.03 ± 0.09 |

### III.4. Survival During Simulation of GI Digestion

The ability of the strain Lacticaseibacillus rhamnosus TOM 22.8 DSM 33500 to survive during gastrointestinal transit (GI) was determined in vitro using simulated gastric juice (SGJ) and simulated intestinal fluid (SIF). In detail, SGJ (0.3% pepsin, 0.5% NaCl, adjusted to pH 2 by adding 1 M HCl) and SIF (0.1% pancreatin, 0.5% biliary salt, 0.5% NaCl, 0.4% phenol, adjusted to pH 8 by adding 1 M NaOH) were prepared immediately before use and sterilised using a 0.22 μm cellulose acetate filter (Minisart, Sartorius, Gottinga filters, Germany). All chemicals were obtained from Sigma Aldrich (St. Louis, Mo.). Cell cultures in exponential growth phase (9 log cfu/ml) were pelleted by centrifugation and resuspended in phosphate buffer (PBS). The cell suspension obtained was mixed with SGJ and incubated for 2 hours at 37° C., under microaerophilic conditions under stirring (200 rpm). The cells, pelleted by centrifugation, were suspended again in SIF and incubated at 37° C. for 3 hours. The cells treated with SGJ and SGJ-SIF were subjected to the plate count of viable cells. The analysis was carried out in triplicate and the results, reported in Table 11 and expressed as log cfu/ml, are reported as mean and standard deviation.

The strain showed excellent survival during simulated gastrointestinal transit under static conditions. During simulated gastrointestinal digestion, a survival greater than 90% was observed after treatment with simulated gastric juice (SGJ, survival (SR) 99%) and simulated intestinal fluid (SIF, SR 97%) (Table 11).

TABLE 11

| Activity tested | | TOM 22.8 |
|---|---|---|
| Survival during simulation | Initial cell density | 9.15 ± 0.03 |
| of GI digestion | Simulated gastric juice (SGJ) | 9.12 ± 0.08 |
| (log cfu/ml) | Simulated intestinal | 8.91 ± 0.07 |
| | fluid (SIF) | |

### III.5. Antimicrobial Activity

The strain Lacticaseibacillus rhamnosus TOM 22.8 DSM 33500 was tested for antagonistic activity using Escherichia coli ATCC 700414, Escherichia coli ATCC 25922, Staphilococcus aureus ATCC 6538, Gardnerella vaginalis ATCC 14018, G. vaginalis ATCC 14019, Candida albicans ATCC 10231, Candida krusei ATCC 14243, Candida glabrata ATCC 90030 and Candida tropicalis ATCC 13803 as target microorganisms. The test was carried out by means of agar spot test. After incubation at 37° C. for 48 hours, antimicrobial activity was evaluated by measuring the inhibition halo obtained. The results, reported in Table 12, were expressed as −(no activity); +(inhibition halo with diameter <10 mm); ++(inhibition halo with diameter comprised between 11 and 20 mm); +++(inhibition halo with diameter >20 mm). The strain under study showed antimicrobial activity against the pathogens tested.

TABLE 12

| | |
|---|---|
| Activity tested | TOM 22.8 |

| | | |
|---|---|---|
| Antimicrobial activity | E. coli ATCC 25922 | +++ |
| | E. coli ATCC 700414 | +++ |
| | G. vaginalis ATCC 14018 | +++ |
| | G. vaginalis ATCC 14019 | ++ |
| | C. albicans ATCC10231 | ++ |
| | C. krusei ATCC 14243 | ++ |
| | C. glabrata ATCC 90030 | ++ |
| | C. tropicalis ATCC 13803 | ++ |
| | S. aureus ATCC 6538 | ++ |

The highest antimicrobial activity was shown against E. coli ATCC 25922, E. coli ATCC 700414, and G. vaginalis ATCC 14018 (inhibition zone larger than 20 mm). Inhibition zones with a diameter comprised between 11 and 20 mm were observed against G. vaginalis ATCC 14019, C. albicans ATCC10231, C. krusei ATCC 14243, C. glabrata ATCC 90030, C. tropicalis ATCC 13803, and S. aureus ATCC 6538.

### III.6. Anti-inflammatory Activity in Macrophages

LX-2 cell lines (human hepatic stellate cells), cell line of monocyte origin derived from neoplastic cells (monocytes differentiated in macrophages and subsequently treated with lipopolysaccharide (LPS) to induce inflammation), used as an in vitro model of inflammation, were suspended in Dulbecco's modified Eagle's medium (DMEM) 1 g/L D-glucose (Gibco, Life Technologies, Milan, Italy) supplemented with 10% w/w bovine serum (FBS) (Invitrogen, Carlsbad, Calif., USA), 1% penicillin/streptomycin (Carlo Erba, Milan, Italy) and 60 mg/mL gentamicin (Gibco). U937 cells were differentiated into macrophages by treating with 200 nM PMA (Phorbol 12-myristate 13-acetate) for 72 h. In order to simulate the inflammatory process, the cells were pre-treated with lipopolysaccharide (LPS) at a concentration of 100 ng/mL for 2 hours. The anti-inflammatory effect of the strains under study was evaluated by treating the differentiated cells with the bacterial strains at the concentration of 10 μg/mL for 6 hours. At the end of the treatment, the cells were washed with PBS, harvested by means of trypsinisation and then lysed for RNA extraction. Quantification of COX-1 (Cyclooxygenase-1), COX-2, IL-8 and IL-10 genes was performed by means of real-time qRT-PCR. Untreated cells were used as control. The analyses were carried out in triplicate and the results are reported as mean and standard deviation.

The analysis carried out show that the strain Lacticaseibacillus rhamnosus TOM 22.8 DSM 33500 has anti-inflammatory activity in macrophages, determining the decrease in pro-inflammatory interleukin IL-8 and the increase in anti-inflammatory interleukin IL-10 (Table 13).

TABLE 13

| Activity tested | | TOM 22.8 |
|---|---|---|
| Antioxidant | COX-1 | 23.15 ± 1.26 |
| activity in | (LPS 2.11 ± 0.10) | |
| macrophages | COX-2 | 0.06 ± 0.03 |
| | (LPS 15.23 ± 0.76) | |
| | IL-8 | 0.54 ± 0.05 |
| | (LPS 0.67 ± 0.03) | |
| | IL-10 | 611.83 ± 25.32 |
| | (LPS 125.50 ± 6.42) | |

### III.7. Antioxidant activity

The strain Lacticaseibacillus rhamnosus TOM 22.8 DSM 33500 was tested for antioxidant activity using 1 mL of supernatant (cell free) added with 1 mL of PBS (0.1 M, pH 7.0) and 1 mL of linoleic acid (50 mM) in ethanol (99.5%). Oxidation was measured by determining ferric thiocyanate. Butylated hydroxytoluene (BHT) and α-tocopherol (1 mg/mL) were used as positive controls. MRS broth was used as negative control. The results, reported in Table 14, were expressed as absorbance values at 500nm. The analysis was carried out in triplicate.

The strain tested showed antioxidant activity. In particular, the strain L. rhamnosus TOM 22.8 was able to combat the peroxidation of linoleic acid showing antioxidant activity similar (P<0.05) to that detected for α-tocopherol. α-tocopherol is used as a control.

TABLE 14

| Activity tested | TOM 22.8 |
|---|---|
| Antioxidant activity (negative control: Abs_{500 nm} = 2; | 0.51 |
| positive control: Abs_{500 nm} = 0.25) | |

### III.8. Auto-Aggregation, Hydrophobicity and Co-Aggregation

One ml of cell suspension (10⁹ cfu/mL) in PBS buffer was vortexed for 10 seconds and incubated at room temperature for 5 hours. Subsequently, an aliquot (0.1 ml) of the upper phase of the suspension was mixed with 500 μl of PBS buffer and absorbance at 600 nm was determined. Auto-aggregation (Auto-A%) was expressed as a percentage decrease in absorbance after 5 h (ΔAbs×100) with respect to that of the original suspension (Abst0) as follows: Auto-A%=ΔAbs/Absₜ₀×100.

Hydrophobicity (H%) was determined by mixing equal volume of xylene and cell suspension in PBS (10⁹ cfu/mL). After incubation at 37° C. for 10 min the suspension was maintained at 37° C. for 5 h in order to obtain the phase separation. The aqueous phase was taken and subjected to determination of absorbance at 600 nm. Hydrophobicity was calculated as percentage decrease in absorbance (ΔAbs×100) of the aqueous phase after incubation with respect to absorbance of the base suspension: H%=ΔAbs/Absₜ₀×100.

Co-aggregation (Co-A%) was evaluated using the strains Escherichia coli 555, Gardnerella vaginalis ATCC 14018, Candida albicans ATCC 10231, and Candida krusei ATCC 14243 as co-aggregation partners. Equal volume (2 mL) of the cell suspension of the strain TOM 22.8 and each of the pathogenic strains were mixed and vortexed for 30 seconds then maintained at room temperature for 5 h. The absorbance at 600 nm of the mixture TOM 22.8+pathogen (Abs_{(TOM22.8+pat)}) and of the strain TOM 22.8 alone (Abs_{TOM22.8}) was determined. Co-aggregation was calculated as follows: Co-A%=((Abs_{CA15}+Absₚₐₜ)/2−Abs_{(CA15+pat)}/Abs_{CA15}+Abspat/2)×100.

The strain Lacticaseibacillus rhamnosus TOM 22.8 DSM 33500 showed good auto-aggregation, hydrophobicity and co-aggregation with the tested pathogens, indirectly showing the ability to adhere to the intestinal mucosa and prevent the development of pathogens (Table 15).

TABLE 15

| | | Co-A % | | | |
|---|---|---|---|---|---|
| | | | G. vaginalis | C. albicans | C. glabrata |
| | | E. coli | ATCC | ATCC | ATCC |
| H % | Auto-A % | 555 | 14018 | 10231 | 14243 |
| 82.12 ± | 66.54 ± | 61.28 ± | 67.35 ± | 61.24 ± | 65.33 ± |
| 0.13 | 0.04 | 0.07 | 0.15 | 0.15 | 0.09 |

### III.9. Production of Hydrogen Peroxide

The ability to produce hydrogen peroxide (H₂O₂) was evaluated by culturing the strain on MRS agar containing 0.25 mg/mL of 3,3′,5,5′-tetramethylbenzidine and 0.01 mg/mL horseradish peroxidase. After incubation under anaerobic conditions for 72 hours, the plates were exposed to air and the production of H202 was evaluated based on the appearance of blue staining. The ability to produce hydrogen peroxide (H202) was evaluated based on the time required for the appearance of the blue coloration, as follows: absent (score 0, no production of blue coloration); low (score 1, time >20 min), average (score 2, time 10-20 min) and high (score 3, time <10 min). The strain Lactobacillus acidophilus ATCC 4356 was used as positive control.

The results, in Table 16, were expressed as −(absence of production) or +(production of H₂O₂).

The strain Lacticaseibacillus rhamnosus TOM 22.8 DSM 33500 showed ability to produce H₂O₂. In particular, TOM 22.8 DSM 33500 can be classified as a high producer of H₂O₂ (score 3) since the time required for the appearance of the blue coloration is less than 10 minutes.

TABLE 16

| Activity tested | TOM 22.8 |
|---|---|
| Production of H₂O₂ | + |

### III.10. Production of Exopolysaccharides (EPS)

It was tested in plate by means of touch loop test. Cell cultures in exponential growth phase were propagated in De Man, Rogosa and Sharpe (MRS) agar (Oxoid) growth medium. After incubation at 37° C. for 48 hours, the EPS production was evaluated based on the presence of mucoid colonies. The results, reported in Table 17, were expressed as: −(no production of EPS); +(production of EPS). The production of exopolysaccharides was determined according to the phenol/sulfuric acid method and the amount produced, expressed in mg/l, was estimated using glucose (50-500 mg/l) as standard. The strain L. rhamnosus TOM 22.8 was able to produce EPS with a value of 273 mg/l.

The strain tested showed ability to produce EPS.

TABLE 17

| Activity tested | TOM 22.8 |
|---|---|
| EPS production | + |

### III.11. Strain Stability after Freeze-Drying and Encapsulation

The strain Lacticaseibacillus rhamnosus TOM 22.8 DSM 33500 was subjected to lyophilisation and encapsulation. The viability of the encapsulated strain was evaluated for 12 months, under freezing and storage conditions at room temperature, by plate counting the viable cells. At the time of encapsulation, the population density of the strain TOM 22.8 was 1.33×10¹⁰.

The strain under study showed good freeze-drying ability and good stability up to 12 months, in capsules placed both at room temperature and under refrigerated conditions (Table 18).

TABLE 18

| STORAGE TIME | STORAGE METHODS | TOM 22.8 (cfu/g) |
|---|---|---|
| 1 MONTH | Room T | 2.0 × 10⁹ |
| | +4° C. | 1.2 × 10¹⁰ |
| 4 MONTHS | Room T | 1.8 × 10⁹ |
| | +4° C. | 1.0 × 10¹⁰ |
| 6 MONTHS | Room T | 6.9 × 10⁸ |
| | +4° C. | 7.4 × 10⁹ |
| 9 MONTHS | Room T | 3.1 × 10⁸ |
| | +4° C. | 4.6 × 10⁹ |
| 12 MONTHS | Room T | 5.6 × 10⁷ |
| | +4° C. | 2.8 × 10⁸ |

Preparation of the capsules and stability test.

The strain L. rhamnosus TOM 22.8 was freeze-dried and encapsulated using the capsule machine model MG2 Supreme (MG2, Bologna, Italy). Each capsule contains 10 log cfu/g of L. rhamnosus TOM 22.8, dry starch (118.9 mg), magnesium stearate (4.05 mg) and silicon dioxide (4.05 mg). The capsules were produced under controlled conditions. The checks were carried out throughout the process to ensure compliance with quality standards based on the legislation in force.

The product complies with the A.Q.L. established for the physical characteristics and it is free from preservatives. Stability tests were carried out quarterly for 18 months to prove that the quality of the active ingredients varies over time under the influence of both temperature and humidity.

In detail, the stability of the capsules during storage for 18 months at 25° C. ±2° C. at 60% ±5% humidity revealed that the product is stable and complies with the regulatory specifications. E. coli, Salmonella spp. and Staphylococcus aureus are totally absent. Furthermore, bile tolerant Gram negatives, total yeasts and moulds and total aerobic microbial count are less than 1 log unit. A high stability of the encapsulated strain of L. rhamnosus TOM 22.8 with a cell density of 10 log cfu/g after 18 months of storage was detected.

### IV: Clinical Study

Study on the effects of the bacterial strains Lacticaseibacillus rhamnosus TOM 22.8 DSM 33500 in the treatment of bacterial vaginitis and/or fungal infections.

The strain Lacticaseibacillus rhamnosus TOM 22. 8 DSM 33500 was subjected to evaluation of the efficacy thereof through an in vivo trial at the Obstetric and Gynaecologic Clinic “Policlinico-Vittorio Emanuele” and directed by Prof. Antonio Cianci, director of the aforementioned clinic. The study provided for a recruitment of 70 female patients with signs and/or symptoms related to vaginal dysbiosis (bacterial and/or fungal) to whom the strain Lacticaseibacillus rhamnosus TOM 22.8 DSM 33500 was administered orally or alternatively through the vaginal route for 10 days.

The inclusion criteria were: presence of at least one vaginal sign or symptom (leucorrhoea, burning sensation, itching and vaginal discomfort), presence of at least 3 criteria of Amsel and a Nugent score ≥7 or a lactobacilli grade ≥2 (LBG), presence of blastospores and/or blastospore pseudo-plasma and/or pseudoips evaluated under microscopy of the fresh sample.

The study excluded all women who had sexually transmitted diseases due to Chlamydia, Neisseria gonorrhoeae, or Trichomonas vaginalis as well as those with specific cervical vaginitis or severe vulvovaginal symptoms related to acute candidiasis; herpes simplex infection; human papillomavirus or human immunodeficiency virus infections; used antibiotics, antimycotics, probiotics or immunosuppressants over the last four weeks; used vaginal contraceptives and had any other physiological or pathological condition that could potentially interfere with the results of the study (pregnancy or breastfeeding, chronic diseases, neoplastic diseases, diabetes, bleeding of the genital tract).

The study was conducted in accordance with current standards of good clinical practice and in accordance with the Helsinki Declaration (2000), the World Medical Association. Women who participated in the study were adequately informed as pertains to the purposes of the study and the procedures carried out and they signed an informed consent prior to recruitment.

A sample of vaginal discharges was collected from each patient, collected at the vaginal fornix at the following times:
T0: At the time of recruitment,
T1: 10 days after initiation of therapy (at the end of the administration of the strain),
T2: one month after the end of therapy.

Vaginal samples were collected using sterile cotton swabs provided with transport medium (Transystem Amies Medium Clear, Biolife, Milan, Italy), immediately transferred, under refrigeration conditions, to the laboratory of the Department of Agriculture, Food and Environment of the University of Catania (Catania, Italy), and subjected to cultural examination, using specific and selective media for the search of the main microbial groups of vaginal origin: Gardnerella vaginalis; Staphylococcus spp.; Candida spp; Escherichia coli; Enterococcus spp.; Streptococcus spp; lactobacilli.

In particular, the following procedures were carried out:
microscopic examination of the morphotype of the bacterial population; the microbial communities and the lactobacilli population, distinguished based on morphology and Gram staining, were evaluated in semi-quantitative terms (absent; scarce+; discrete++; abundant+++);
evaluation of the fungal population;
evaluation of the possible presence of “clue cells”;
determination of the score obtained from the ratio between the various bacterial morphotypes (lactobacilli, Gardnerella vaginalis, Mobiluncus spp., anaerobic).

All analyses were carried out in triplicate and the results are reported as log cfu/ml mean and standard deviation.

In a first phase of said clinical study (preliminary phase), the following groups of patients were examined:
10 patients not treated with the strain TOM 22.8 (control),
10 patients treated with the strain TOM 22.8 through the topical route,
10 patients treated with the strain TOM 22.8 through the oral route.

The preliminary results obtained from said first phase of the clinical study (Table 19) showed a significant improvement in clinical symptoms and a decrease in the indexes related to the dysbiosis condition in patients treated with the strain TOM 22.8 compared to untreated patients.

In particular, during the entire period of administration a decrease in the Nugent score, the Whiff test (amine odour test % positivity) and in the vaginal pH which returned to the physiological value (pH 4.5), as reported in Table 19, was observed.

From the microbiological point of view, there was a significant decrease in microbial species associated with bacterial dysbiosis, such as Gardnerella vaginalis, E. coli, Streptococcus spp., a concomitant increase in the lactobacilli population, as well as a significant decrease in fungal infection.

TABLE 19

| | Collection | Nugent score | | | Whiff | pH > |
|---|---|---|---|---|---|---|
| Patients | time | ≤3 | 4-6 | ≥7 | test | 4.5 |
| N = 20 | T0 | 0 | 0 | 20 | 100% | 20 |
| | T1 | 18 | 2 | 0 | 2% | 2 |
| | T2 | 18 | 2 | 0 | 4% | 2 |
| N* = 10 | T0 | 0 | 0 | 10 | 100% | 10 |
| | T1 | 0 | 0 | 10 | 98% | 10 |
| | T2 | 0 | 1 | 9 | 93% | 10 |

N: treated patients; N*: control patients.

Table 20 shows the microbial count values expressed in log cfu/ml of vaginal exudate at the time of recruitment (T0), after 10 days of oral treatment (OS) with the strain T0M22.8 (T1) and after 1 month from the end of treatment (T2).

Table 21 shows the microbial count values expressed in log cfu/ml of vaginal exudate at the time of recruitment (T0), after 10 days (T1) of topical treatment with the strain T0M22.8 and after 1 month (T2) from the end of treatment.

Lastly, Table 22 shows the microbial count values expressed in log cfu/ml of vaginal exudate of the subjects allocated in the control group (wait and see).

TABLE 20

| | | | | | Gardnerella | Candida | Candida | Candida | Escherichia |
|---|---|---|---|---|---|---|---|---|---|
| | Lactobacilli | Enterococci | Staphylococci | Streptococci | vaginalis | glabrata | albicans | krusei | coli |
| T0 | 3.85 ± 0.22 | 1.70 ± 0.16 | <1 | <1 | 4.20 ± 0.21 | 5.60 ± 0.22 | 3.40 ± 0.21 | 3.74 ± 0.18 | 3.30 ± 0.28 |
| T1 | 6.95 ± 0.19 | 0.52 ± 0.21 | <1 | <1 | 1.06 ± 0.33 | 1.51 ± 0.24 | 0.27 ± 0.31 | 1.24 ± 0.1 | 1.07 ± 0.14 |
| T2 | 8.23 ± 0.12 | 0.51 ± 0.03 | <1 | <1 | 1.03 ± 0.09 | 1.32 ± 0.06 | 0.11 ± 0.03 | 1.14 ± 0.16 | 1.05 ± 0.09 |
| *P T0vsT1 | 0.000118 | 0.001677 | — | — | 0.000384 | 5.6E−05 | 5.538−05 | 2.93E−05 | 0.000548 |
| *P T0vsT2 | 1.71E−05 | 0.000395 | — | — | 4.11E−05 | 9.98E−06 | 2.47E−05 | 6.28E−05 | 0.000413 |
| *P T1vsT2 | 0.001178 | 0.698066 | — | — | 0.970612 | 0.386837 | 0.174456 | 0.399079 | 0.872158 |

Statistical significance: *P < 0.05

TABLE 21

| | | | | | Gardnerella | Candida | Candida | Candida | Escherichia |
|---|---|---|---|---|---|---|---|---|---|
| | Lactobacilli | Enterococci | Staphylococci | Streptococci | vaginalis | glabrata | albicans | krusei | coli |
| T0 | 2.95 ± 0.25 | <1 | <1 | <1 | <1 | 4.16 ± 0.21 | 4.81 ± 0.20 | 4.41 ± 0.15 | <1 |
| T1 | 5.80 ± 0.17 | <1 | <1 | <1 | <1 | 0.8 ± 0.22 | 1.2 ± 0.29 | 1.03 ± 0.08 | <1 |
| T2 | 5.90 ± 0.11 | <1 | <1 | <1 | <1 | 0.12 ± 0.03 | 1.28 ± 0.01 | 1.06 ± 0.13 | <1 |
| *P T0vsT1 | 0.000194 | — | — | — | — | 0.000101 | 0.000139 | 9.07E−06 | — |
| *P T0vsT2 | 0.000116 | — | — | — | — | 1.19E−05 | 1.79E−05 | 1.75E−05 | — |
| *P T1vsT2 | 0.530463 | — | — | — | — | 0.010283 | 0.729672 | 0.796653 | — |

Statistical significance: *P < 0.05

TABLE 22

| | | | | | Gardnerella | Candida | Candida | Candida | Escherichia |
|---|---|---|---|---|---|---|---|---|---|
| | Lactobacilli | Enterococci | Staphylococci | Streptococci | vaginalis | glabrata | albicans | krusei | coli |
| T0 | 4.02 ± 0.71 | 4.14 ± 0.77 | 2.11 ± 1.69 | 4.07 ± 1.32 | 4.27 ± 0.36 | 3.61 ± 1.52 | 3.65 ± 1.30 | 2.90 ± 1.51 | 1.83 ± 1.26 |
| T1 | 4.18 ± 0.87 | 4.38 ± 0.94 | 2.37 ± 1.59 | 4.23 ± 0.98 | 4.34 ± 0.37 | 3.85 ± 1.74 | 3.78 ± 1.37 | 3.62 ± 1.91 | 2.50 ± 1.50 |
| T2 | 3.87 ± 0.89 | 4.78 ± 0.89 | 2.42 ± 1.83 | 4.61 ± 1.15 | 4.40 ± 0.27 | 4.04 ± 1.77 | 3.92 ± 1.42 | 3.64 ± 1.91 | 2.68 ± 1.52 |
| *P T0vsT1 | 0.66379 | 0.561081 | 0.737189 | 0.771997 | 0.658179 | 0.763677 | 0.847928 | 0.384905 | 0.317805 |
| *P T0vsT2 | 0.713824 | 0.135989 | 0.712038 | 0.369320 | 0.373968 | 0.588315 | 0.682609 | 0.372584 | 0.212682 |
| *P T1vsT2 | 0.466611 | 0.385586 | 0.953394 | 0.465672 | 0.697889 | 0.818336 | 0.829122 | 0.983383 | 0.805061 |

Statistical significance: *P < 0.05

1. A composition comprising a bacterial strain belonging to the species Lacticaseibacillus rhamnosus identified as Lacticaseibacillus rhamnosus TOM 22.8 and deposited at Deutsche Sammlung von Mikroorganismen and Zellkulturen GmbH (DSMZ) under deposit number DSM 33500.

2. (canceled)

3. A method for the preventive or curative treatment of a genitourinary infection and/or inflammation, and of associated disorders or symptoms, in a male or female subject in need comprising administering the composition of claim 1 to the subject.

4. The method according to claim 3, wherein said genitourinary infection and/or inflammation, preferably of the genital tract, and the associated disorders or symptoms are selected from: bacterial and/or fungal vaginitis, vaginal (or vulvovaginal) candidiasis , bacterial vaginitis, atrophic vaginitis, male genital bacterial and/or fungal infections, balanitis, posthitis and balanoposthitis, pain and/or itching in the genital area, dysuria, strangury, malodorous vaginal discharge, vaginal dryness, and dyspareunia; wherein said infection or inflammation is acute or chronic.

5. The method according to claim 3, wherein said genitourinary infection and/or inflammation is a vaginal infection and/or inflammation of bacterial and/or fungal origin; preferably selected from bacterial and/or fungal vaginitis, vaginal candidiasis and bacterial vaginosis.

6. The method according to claim 3, wherein said genitourinary infection and/or inflammation, preferably of the urinary tract, and the associated disorders or symptoms are selected from: bacterial and/or fungal urinary tract infections (UTI) or of the lower urinary tract, cystitis, urethritis, prostatitis, lower urinary tract infections in subjects with bladder catheters, dysuria and painful urination; wherein said infection or inflammation is acute or chronic.

7-8. (canceled)

9. The composition A composition according to claim 1, and at least one pharmaceutical or food grade additive and/or excipient.

10. The composition of claim 9, wherein said composition further comprises at least one prebiotic; preferably said at least one prebiotic is selected from: inulin, fructooligosaccharide (FOS), galactooligosaccharide (GOS), xylooligosaccharide (XOS), guar gum and mixtures thereof; more preferably inulin.

11. The composition according to claim 9, wherein said composition is formulated for oral use; preferably in solid form.

12. The composition according to claim 9, wherein said composition is formulated for topical use; preferably for topical vaginal or genital use.

13. The composition according to claim 9 for use as medicament.

14. The composition according to claim 9, for use in a method for the preventive or curative treatment of a genitourinary infection and/or inflammation, and of associated disorders or symptoms, in a male or female subject in need; preferably in a female subject.

15. The composition for use according to claim 14, wherein said genitourinary infection and/or inflammation, preferably of the genital tract, and the associated disorders or symptoms are selected from: bacterial and/or fungal vaginitis, vaginal (or vulvovaginal) candidiasis , bacterial vaginitis, atrophic vaginitis, male genital bacterial and/or fungal infections, balanitis, posthitis and balanoposthitis, pain and/or itching in the genital area, dysuria, strangury, malodorous vaginal discharge, vaginal dryness, and dyspareunia; wherein said infection or inflammation is acute or chronic.

16. The composition for use according to claim 14, wherein said genitourinary infection and/or inflammation is a vaginal infection and/or inflammation of bacterial and/or fungal origin; preferably selected from bacterial and/or fungal vaginitis, vaginal candidiasis and bacterial vaginosis.

17. The composition for use according to claim 14, wherein said genitourinary infection and/or inflammation, preferably of the urinary tract, and the associated disorders or symptoms are selected from: bacterial and/or fungal urinary tract infections (UTI) or of the lower urinary tract, cystitis, urethritis, prostatitis, lower urinary tract infections in subjects with bladder catheters, dysuria and painful urination; wherein said infection or inflammation is acute or chronic.
